(19) United States
(12) Patent Application Publication
(10) Pub. No.: US 2009/0093691 A1
(43) Pub. Date: Apr. 9, 2009

(54) DEVICE FOR MONITORING THE STATUS OF A PATIENT AND TREATMENT BASED THEREUPON

(75) Inventors: Hendrika Cecilia Krijnsen, Boxtel (NL); Geert Langereis, Eindhoven (NL); Michel Paul Barbara Van Bruggen, Helmond (NL); Ventzeslav Petrov Iordanov, Valkenswaard (NL)

Correspondence Address: PHILIPS INTELLECTUAL PROPERTY & STANDARDS, P.O. BOX 3001, BRIARCLIFF MANOR, NY, 10510, US

(73) Assignee: KONKLIJKE PHILIPS ELECTRONICS N.V., EINDHOVEN (NL)

(21) Appl. No.: 12/278,978

(22) PCT Filed: Jan. 31, 2007
(86) PCT No.: PCT/IB2007/050328

§ 371 Date: Aug. 11, 2008

(30) Foreign Application Priority Data
Feb. 9, 2006 (EP) 06101438.7

(51) Int. Cl. A61M 36/12; A61B 5/07; A61B 5/00

(52) U.S. Cl. 600/302; 600/300; 600/7

(57) ABSTRACT

The invention relates to a device for drug administration and/or monitoring the status of a patient, said device comprising at least one measuring means for measuring at least one body parameter of a patient for at least ≧1 measuring cycle, the measuring means being located in or in the vicinity of a bio-implantable and/or bio-implementable needle.

This invention is in the field of devices for drug and/or radiotherapy administration.

Most biological functions and processes are anything but constant. Instead, often a prominent, genetically based time structure consisting of rhythms is observed.

The concept of homeostasis stipulates that there is constancy of the endogenous compounds in blood. This is a most powerful construct in biology, which has influenced not only the teaching and understanding of the medical sciences but also the practice of clinical medicine. According to this concept, the risk of the occurrence and exacerbation of disease is independent of the time of day, day of month, and month of year, as is the response of patients to diagnostic tests and medications. However, findings from the field of biologic rhythm study (chronobiology) challenge the concept of homeostasis, as well as many of the assumptions and procedures of clinical medicine.

Many biological functions wax and wane in cycles that repeat on a daily, monthly

or annual basis. Such patterns do not reflect simply an organism's passive response to environmental changes, such as daily cycles of light and darkness. Rather, they reflect the organism's biological rhythms, that is, its ability to keep track of time and to direct changes in function accordingly.

However, in some fields the oscillations in the biological functions are only quite small and have to be measured quite accurately in order to make proper use of the patterns.

It is therefore an object of the present invention to provide a device for monitoring the status of a patient which is capable of measuring changes in body parameter(s) of a patient in an accurate and reliable way.

This object is solved by a device according to claim 1 of the present invention. Accordingly, a device for drug administration and/or monitoring the status of a patient is provided, comprising at least one measuring means, which measures at least one body parameter of a patient for at least ≧1 measuring cycle, the measuring means being located in or in the vicinity of a bioimplantable and/or bioimplementable needle.

As a result, at least one of the following advantages is achieved for most of the applications within the present invention.
By measuring temperature and/or pressure one can monitor in real-time the tumor rhythm, enabling better treatment and choosing the most appropriate time for applying the therapy/medicines.
There are several sensors along the length, which allow instantaneous differential measurements of e.g. temperature or pressure inside and outside the tumor. The tumor boundaries can be defined as well.
By measuring temperature and/or pressure one can monitor in real-time the growth or progression of the tumor, based upon which the treatment frequency can be adjusted.
Due to the fact that the device comprises a bioimplantable and/or bioimplementable needle, the device can be brought much closer to e.g. a tumor inside a patient, thus allowing a much higher efficiency in measuring e.g. the tumor temperature.
The very same device can be used for both therapeutic and monitoring purposes, separately or simultaneously.

The term “measuring cycle” means and/or includes especially that a body parameter of the patient is measured which is known to behave in a cyclic and/or periodic manner, e.g. the body temperature. A measuring cycle in the sense of the present invention may last a day (circadian), however, also longer (e.g. infradian) and/or shorter cycles (ultradian) are feasible as well as embodiments of the present invention.

The term “bioimplantable and/or bioimplementable needle” means and/or includes especially that a needle can exist in an in vivo environment (within living tissue) and perform intended functions for a period of time with no detrimental effect on the patient.

According to an embodiment of the present invention, the measuring means is located in or in the vicinity of the tip of the bioimplantable and/or bioimplementable needle.

According to an embodiment of the present invention, the at least one body parameter includes body temperature, core body temperature, skin surface temperature, tumor temperature, tumor interstitial fluid pressure, blood pressure, interstitial fluid pressure, melatonin level, triacylglycerol level, cortisol level.

According to an embodiment of the present invention, the at least one measuring means forms an integral part of the needle.

The term “integral part” means and/or includes especially that the at least one measuring means is fully inserted in the needle, preferably close to the tip, or even form the tip of the needle. According to a different embodiment, the needle may be equipped with one or more recesses to insert the measuring means therein.

According to a different embodiment of the present invention, several measuring means are provided at different positions along the needle.

According to a different embodiment of the present invention, the average distance between each of the measuring means is ≧0.1 mm and ≦10 mm.

The measuring means may be spaced longitudinally and/or radially from each other.

According to a different embodiment of the present invention, the average distance between each of the measuring means is ≧0.5 mm and ≦2 mm.

According to a different embodiment of the present invention, the average distance between each of the measuring means is ≧1 mm and ≦1.5 mm.

According to an embodiment of the present invention, the length: width ratio of the needle is ≧8:1 and ≦200:1.

It should be noted that in case the needle is not somewhat cylinder-shaped or does not have a uniform cross section, the term “width” is to be understood as the largest dimension when seen in cross section.

According to an embodiment of the present invention, the length:width ratio of the needle is ≧10:1 and ≦100:1.

According to an embodiment of the present invention, the length:width ratio of the needle is ≧15:1 and ≦60:1.

According to an embodiment of the present invention, the length:width ratio of the needle is ≧25:1 and ≦50:1.

According to an embodiment of the present invention, the needle is essentially made out of a material selected from the group comprising
metals, preferably selected out of the group comprising titanium, titanium-based alloys, austenitic stainless steel, cobalt-chromium and cobalt-based alloys, gold, platinum and mixtures thereof,
ceramics and glasses preferably selected out of the group comprising alumina and zirconia-based glasses and mixtures thereof,
molding plastics, preferably selected out of the group comprising macrolone, polystyrene, PMMA and mixtures thereof,
polymers, preferably selected out of the group comprising polyethylene, polypropylene, Teflon, polyvinyl chloride and mixtures thereof,
   and mixtures thereof.

According to an embodiment of the present invention, the needle is at least partially or wholly covered with a coating made out of a biocompatible material.

According to an embodiment of the present invention, the biocompatible material is selected out of the group comprising silicone and parylene, polymers, preferably polyethylene glycol, metals, advanced ceramics, natural materials, pyrolytic carbon and mixtures thereof.

According to an embodiment of the present invention, the needle is equipped with at least one means for wireless communication. According to an embodiment of the present invention, the needle is adapted to be implanted inside the body of a patient.

According to an embodiment of the present invention, the needle serves as an antenna for wireless communication.

According to an embodiment of the present invention, the needle has a minimum length of ≧7 cm and ≦25 cm.

According to an embodiment of the present invention, the needle has a minimum length of ≧17 cm and ≦20 cm.

According to an embodiment of the present invention, the device comprises at least one drug delivering means adapted to release drugs via the needle.

According to an embodiment of the present invention, the needle comprises at least one conduit for delivering drugs therethrough.

According to an embodiment of the present invention, the conduit is linked to the outside of the needle; according to a different embodiment, the conduit is closed inside the needle.

According to an embodiment of the present invention, the device comprises at least one radioactive seeds-holding means adapted to send radioactive waves via the needle.

According to an embodiment of the present invention, the needle comprises at least one conduit for delivering radioactive seeds therein.

According to an embodiment of the present invention, the at least one conduit of the needle has a diameter of ≧1 mm and ≦5 mm.

According to an embodiment of the present invention, the at least one conduit of the needle has a diameter of ≧0.5 mm and ≦2 mm.

According to an embodiment of the present invention, the at least one conduit of the needle has a diameter of ≧0.01 mm and ≦1 mm.

A device according to the present invention may be of use in a broad variety of systems and/or applications, amongst them one or more of the following:
medical devices for the administration of drugs or radiotherapy
medical devices for treatment of chronic diseases.

The aforementioned components, as well as the claimed components and the components to be used in accordance with the invention in the described embodiments, are not subject to any special exceptions with respect to their size, shape, material selection and technical concept, so that the selection criteria known in the pertinent field can be applied without limitations.

Additional details, features, characteristics and advantages of the object of the invention are disclosed in the subclaims, the Figures and the following description of the respective Figures, tables and examples, which—in an exemplary fashion—show several embodiments of a device as well as a device according to the invention:

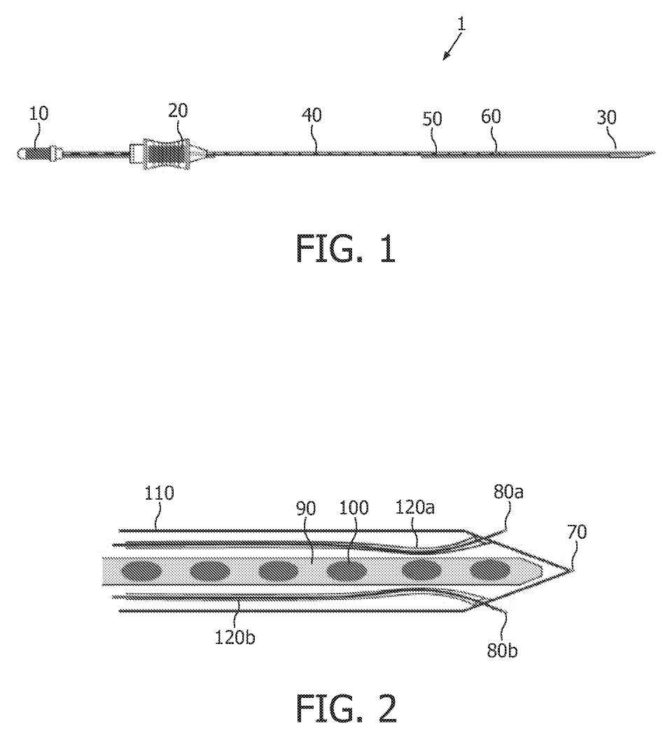

FIG. 1 shows a very schematic side view of a needle for a device according to a first embodiment of the present invention;

FIG. 2 shows a very schematic longitudinal cut-out view of the tip section of a needle for a device according to a second embodiment of the present invention;

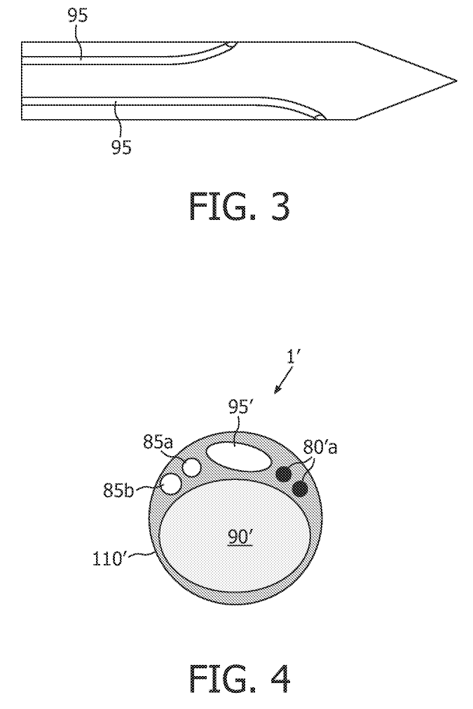

FIG. 3 shows a very schematic longitudinal cut-out view of the tip section of a needle for a device according to a third embodiment of the present invention;

FIG. 4 shows a very schematic cross-sectional view of a needle for a device according to a fourth embodiment of the present invention.

FIG. 1 shows a very schematic side view of a needle 1 for a device according to a first embodiment of the present invention. In this embodiment, the needle is to be implanted inside the body of a patient. Therefore the needle has a length of ≧7 cm and ≦25 cm, as described above, in order to serve as an antenna to communicate with other parts of the device (not shown) and to transmit data. Two further communication means 10 and 20 are provided in the rear section of the needle. The needle furthermore is provided with several temperature sensors 30, 40, 50, 60, which are provided close to the tip (temperature sensor 30) and along the needle (temperature sensors 40-60). The distance between the sensors is ≧0.1 mm and ≦10 mm as described above.

FIG. 2 shows a very schematic longitudinal cross-sectional view of the tip section of a needle for a device according to a second embodiment of the present invention. In the needle body 110 there are three conduits 90, 120a and 120b, of which the conduits 120a and 120b serve as a delivery means for the temperature sensors 80a, 80b, which are present on top of thin wires. The conduits 120a and 120b in this embodiment extend to very close to the tip 70 of the needle in order to collect the temperature data as precisely as possible. The conduit 90 can be filled with several radioactive seeds 100, which serve e.g. to treat a malignant tumor.

FIG. 3 shows a very schematic longitudinal cross-sectional view of the tip section of a needle for a device according to a third embodiment of the present invention. Here, conduits 95 are present, which serve as pathways to a pressure sensor.

FIG. 4 shows a very schematic cross-sectional view of a needle 1′ for a device according to a fourth embodiment of the present invention. In body 110′ of this needle 1′ several conduits 80′a, 85a, 85b, 90′, 95′ are present. The conduits 80′ a serve as conduits for temperature sensors, similar to the needle shown in FIG. 2, and the conduit 95′ serves as a guide to a pressure sensor, similar to the needle shown in FIG. 3. The conduit 90′ is provided with radioactive seeds (not shown), similar to FIG. 2. However, in this embodiment, two more conduits 85a, 85b are present, which serve to deliver other drugs to the outside, e.g. to a tumor.

The particular combinations of elements and features in the above-described embodiments are exemplary only; the interchanging and substitution of these teachings with other teachings in this and the incorporated-by-reference patents/applications are also expressly contemplated. As those skilled in the art will recognize, variations, modifications, and other implementations of what is described herein may occur to those of ordinary skill in the art without departing from the spirit and the scope of the invention as claimed. Accordingly, the foregoing description is by way of example only and is not intended to be construed in a limiting sense. The invention's scope is defined in the following claims and the equivalents thereto. Furthermore, reference signs used in the description and claims do not limit the scope of the invention as claimed.

1. A device for drug administration and/or monitoring the status of a patient comprising at least one measuring means, which measures at least one body parameter of a patient for at least ≧1 measuring cycle, the measuring means being located in or in the vicinity of a bioimplantable and/or bioimplementable needle.

2. The device according to claim 1, wherein the at least one body parameter includes body temperature, core body temperature, skin surface temperature, tumor temperature, tumor interstitial fluid pressure, blood pressure, interstitial fluid pressure, melatonin level, triacylglycerol level, cortisol level.

3. The device according to claim 1, wherein the at least one measuring means forms an integral part of the needle.

4. The device according to claim 1, wherein the length:width ratio of the needle is ≧4:1 and ≦50:1.

5. The device according to claim 1, wherein the needle is essentially made out of a material selected from the group comprising
metals, preferably selected out of the group comprising titanium, titanium-based alloys, austenitic stainless steel, cobalt-chromium and cobalt-based alloys, gold, platinum and mixtures thereof,
ceramics and glasses preferably selected out of the group comprising alumina and zirconia-based glasses and mixtures thereof,
molding plastics, preferably selected out of the group comprising macrolone, polystyrene, PMMA and mixtures thereof,
polymers, preferably selected out of the group comprising polyethylene, polypropylene, Teflon, polyvinyl chloride and mixtures thereof,
and mixtures thereof.

6. The device according to claim 1, wherein the needle is covered with a coating made out of a biocompatible material, preferably a material selected out of the group comprising silicone and parylene, polymers, preferably polyethylene glycol, metals, advanced ceramics, natural materials, pyrolytic carbon and mixtures thereof.

7. The device according to claim 1, wherein the needle is equipped with a means for wireless communication.

8. The device according to claim 1, wherein the needle serves as an antenna for wireless communication.

9. The device according to claim 1, wherein the device comprises at least one drug delivering means adapted to release drugs via the needle and/or the device comprises at least one radioactive seeds-holding means adapted to send radioactive waves via the needle.

10. A system incorporating a device according to claim 1 and being used in one or more of the following applications:
medical devices for the administration of drugs or radiotherapy
medical devices for treatment of chronic diseases.
